(19) United States
(12) Patent Application Publication
(10) Pub. No.: US 2008/0140212 A1
(43) Pub. Date: Jun. 12, 2008

(54) ELONGATED FEMORAL COMPONENT

(76) Inventors: Robert Metzger, Wakarusa, IN (US); Troy W. Hershberger, Winona Lake, IN (US); Thomas Donaldson, Redlands, CA (US)

Correspondence Address: HARNESS, DICKEY & PIERCE, P.L.C., P.O. BOX 828, BLOOMFIELD HILLS, MI, 48303, US

(21) Appl. No.: 11/934,220

(22) Filed: Nov. 2, 2007

Related U.S. Application Data
(63) Continuation-in-part of application No. 10/614,550, filed on Jul. 7, 2003, now abandoned.
Application No. 10/614,550 is a continuation-in-part of application No. 09/855,939, filed on May 15, 2001, now Pat. No. 6,589,283.

(51) Int. Cl. A61F 2/38

(52) U.S. Cl. 623/20.31; 623/20.27; 623/20.14

(57) ABSTRACT

A plurality of knee joint prostheses comprise a first femoral implant and a second femoral implant. The first femoral implant has a first maximum interior anterior to posterior dimension, a first maximum overall exterior anterior to posterior dimension, a first lateral condyle, and a first medial condyle. The second femoral implant has a second maximum interior anterior to posterior dimension that is substantially the same as the first maximum interior anterior to posterior dimension, a second maximum overall exterior anterior to posterior dimension that is different than the first maximum overall exterior anterior to posterior dimension, a second lateral condyle having a medial to lateral dimension that is substantially the same as a medial to lateral dimension of the first lateral condyle, and a second medial condyle having a medial to lateral dimension that is different than a medial to lateral dimension of the first medial condyle.

### CROSS-REFERENCE TO RELATED APPLICATION

This application is a continuation-in-part of U.S. patent application Ser. No. 10/614,550 filed on Jul. 7, 2003, which is a continuation-in-part of U.S. patent application Ser. No. 09/855,939, which was filed on May 15, 2001 and issued as U.S. Pat. No. 6,589,283 on Jul. 8, 2003. The disclosure of the above applications are incorporated herein by reference.

### BACKGROUND OF THE INVENTION

1. Field of the Invention

The present invention relates generally to a knee joint prosthesis, which replaces the articulating knee portion of the femur and tibia, and more particularly, to a knee joint prosthesis system, which includes elongated femoral components for posterior stabilized and fully constrained applications.

2. Discussion of the Related Art

The knee joint is a complex articulating structure. The knee joint includes a femur, which articulates with a tibia, and a patella, which acts as a protective shield for the articulating knee joint. The knee joint also includes soft tissue ligaments, which extend on the medial and lateral side of the knee joint, which are generally referred to as collateral ligaments and ligaments which cross within the knee joint generally referred to as an anterior cruciate ligament and a posterior cruciate ligament.

A knee joint prosthesis typically comprises a femoral component and a tibial component. The femoral component and the tibial component are designed to be surgically attached to the distal end of the femur and the proximal end of the tibia, respectively. The femoral component is further designed to cooperate with the tibial component in simulating and articulating motion of an anatomical knee joint.

The motion of a natural knee joint is kinematically complex. During a relatively broad range of flexion and extension, the articular or bearing surfaces of a natural knee experience rotation, medial and lateral angulation, translation in the sagittal plane, rollback and sliding. Knee joint prostheses, in combination with ligaments and muscles, attempt to duplicate this natural knee motion, as well as absorb and control forces generated during the full range of flexion. Depending on the degree of damage or deterioration of the knee tendons and ligaments, it may be necessary for a knee joint prosthesis to eliminate one or more of these motions in order to provide adequate stability.

To provide for these stabilities, knee joint prostheses generally have different levels of constraint. For example, cruciate retaining (CR) knee joint prostheses provide the least level of constraint, posterior stabilized (PS) knee joint prostheses provide an intermediate level of constraint, while fully constrained knee joint prostheses provide the highest level of constraint upon the kinematic motions of a knee joint. In some situations, a patient may initially require a less constrained knee joint, such as a cruciate retaining (CR) or posterior stabilized (PS) knee joint prosthesis. The patient may later require a fully constrained knee joint prosthesis because the patient is exhibiting instability. Moreover, during the surgical procedure, a surgeon may initially wish to implant a cruciate retaining type knee joint prosthesis and subsequently realize that a posterior stabilized or a fully constrained knee joint prosthesis is required, which may lead to additional surgical resections, as well as surgical time and cost.

There is much debate among knee surgeons regarding whether to resect the posterior cruciate ligament (PCL). Some surgeons will preserve the PCL if at all possible. Many other surgeons will selectively preserve the PCL. Some surgeons who sacrifice the PCL may rely on flexion-extension balance and tibial inserts for stability. Other surgeons may advocate PCL excision and substitute for the ligament using a posterior stabilized (PS) knee joint prosthesis.

PCL substitution with a posterior stabilized (PS) knee joint prosthesis generally requires the removal of intercondylar bone for the PS knee joint prosthesis. As a consequence, there is increased bone removal compared with PCL preservation and the use of a cruciate retaining CR knee joint prosthesis. It has also been suggested in the literature that removal of intercondylar bone may predispose to intercondylar bone fracture. Additionally, resection of the PCL results in an increase in flexion gap relative to extension gap. In contrast to this, the surgical technique for PCL retention is sometimes less forgiving than for PCL substitution because, in addition to balancing the flexion and extension gaps and collateral ligaments, balancing of the PCL is required. In some patients, the PCL may be absent or incompetent and balancing is, therefore, difficult to achieve.

Despite the PCL debate, there appears to be no clear advantage to PCL preserving or substituting designs with regard to knee joint prosthesis. Clinical results appear to indicate that success of knee joint prostheses is associated with factors other than whether or not the PCL is preserved.

Normally, a surgeon will preoperatively select a certain type of knee joint prosthesis. However, the surgeon may find interoperatively in trial, that the preselected prosthesis may not provide the best function of the knee joint and that surgical adjustments in the knee joint are required. In this case, the surgeon is faced with several disadvantages, which will be illustrated in the examples below.

In one example, the surgeon may decide preoperatively to use a posterior stabilized PS knee joint prosthesis. Then, interoperatively in trial if the surgeon determines that the increase in flexion gap is minimal, the surgeon may decide not to compensate for the increased flexion gap. However, if the increase in flexion gap is greater than the surgeon had interoperatively evaluated it to be, the knee joint may postoperatively become loose in flexion which can affect the stability and balance of the implanted knee joint. Additionally, the remaining ligaments in the patients knee joint may become lax and this can also affect stability and wear in the prosthetic knee joint. This is because there may be more edge loading or rotation in the bearing of the prosthetic knee joint and that may be disadvantageous for the knee joint prosthesis patient in the long term.

In another situation, the surgeon may decide interoperatively to change from a cruciate retaining (CR) knee joint prosthesis to a posterior stabilized (PS) knee joint prosthesis. However, by removing the PCL, the flexion gap will increase and further bone resection may be required. To address this situation, the surgeon may opt to use a larger femoral component. However, the larger femoral component places the articulation point of the knee joint in the same position relative to the anterior cortex and by using the larger femoral component, it will occupy the region of the extra flexion gap. But the width in the medial-lateral (ML) portion increases with the larger femoral component. This can restrict flexion of the knee, which might not be acceptable to an active knee joint prosthesis patient.

In still another example, where the surgeon decides to interoperatively change from a cruciate retaining (CR) knee joint prosthesis to a posterior stabilized (PS) knee joint prosthesis and the femur has already been resected to form the engagement surface, the surgeon will generally be unable to use a larger femoral member without using a posterior augment. Currently, many surgeons are reluctant to use a posterior augment in the knee for fear of future deterioration of the knee joint. Additionally, if there is an increase in flexion gap, the surgeon may decide to insert a thicker bearing to decrease the flexion gap. The end result is that in extending the knee position, the patient may complain of a “tight-knee.” Anticipating this, a surgeon may resect the femoral distal bone and move the femoral component up the leg to obtain an equal gap in flexion and extension. However, this may create problems with patella tracking in the knee joint and the knee joint prosthesis patient may find this to be disadvantageous.

In yet an additional example, the patient's femur may be of a width, in the medial-lateral direction (ML), that is wider or narrower than the width, in the medial-lateral direction, of the femoral component. In some patients, the increased ML width may be due to the presence of a medial condyle that is disproportionately wider than the patient's lateral condyle. Such disproportionately wider lateral condyles are typically found in male patients with wide, “squatty” femurs. The use of a femoral component having a ML width that does not at least approximately match the ML width of the femur is undesirable as it can restrict flexion of the knee. Conventional knee joint prostheses do not provide multiple femoral components of varying ML widths to match femurs having varying ML widths, including femurs that have a disproportionally wide medial condyle. This variation in ML widths typically occurs between different sized patients and the male and female anatomy.

What is needed then is a knee joint prosthesis system which does not suffer from the above mentioned disadvantages. This, in turn, will permit interoperative options for selecting a cruciate retaining (CR) knee joint prosthesis, a posterior stabilized (PS) knee joint prosthesis or a fully constrained knee joint prosthesis where the posterior stabilized and the fully constrained knee joint prosthesis compensate for the increased flexion gap due to resection of the posterior cruciate ligament, reduce or eliminate the requirement for further bone resection irrespective of the type of constraint knee joint prosthesis selected, thereby reducing surgical time, cost and complexity, and further provide selection between the various constrained knee joint prostheses without having to oversize or undersize any of the particular components of the knee joint prosthesis. Further, the improved knee joint prosthesis system will include multiple femoral components each having different ML widths, but constant anterior to posterior dimensions, to accommodate femurs of various different ML widths. To accommodate femurs having a disproportionately wide medial condyle, the system will include multiple femoral components each having different ML widths on a medial side only, but constant anterior to posterior dimensions. It is, therefore, an object of the present invention to provide an elongated femoral component in a knee joint prosthesis system that achieves the above-identified advantages.

### SUMMARY OF THE INVENTION

In one embodiment, a plurality of knee joint prostheses comprise a first femoral implant and a second femoral implant. The first femoral implant has a first maximum interior anterior to posterior dimension, a first maximum overall exterior anterior to posterior dimension, a first lateral condyle, and a first medial condyle. The second femoral implant has a second maximum interior anterior to posterior dimension that is substantially the same as the first maximum interior anterior to posterior dimension, a second maximum overall exterior anterior to posterior dimension that is different than the first maximum overall exterior anterior to posterior dimension, a second lateral condyle having a medial to lateral dimension that is substantially the same as a medial to lateral dimension of the first lateral condyle, and a second medial condyle having a medial to lateral dimension that is different than a medial to lateral dimension of the first medial condyle.

Another embodiment provides for a method for implanting a femoral implant of a knee joint prosthesis system having a set of femoral implants to a femur having a resected engagement surface. The method includes resecting the femur to provide the resected engagement surface; providing a first femoral implant having a first maximum interior anterior to posterior dimension, a first maximum overall exterior anterior to posterior dimension, a first lateral condyle, and a first medial condyle; providing a second femoral implant having a second maximum interior anterior to posterior dimension that is substantially the same as the first maximum interior anterior to posterior dimension, a second maximum overall exterior anterior to posterior dimension that is different than the first maximum overall exterior anterior to posterior dimension, a second lateral condyle having a medial to lateral dimension that is substantially the same as a medial to lateral dimension of the first lateral condyle, and a second medial condyle having a medial to lateral dimension that is different than a medial to lateral dimension of the first medial condyle; and securing either the first femoral implant or the second femoral implant to the femur.

Another embodiment provides for a plurality of femoral implants for use in replacing a damaged knee joint comprising a first femoral implant and a second femoral implant. The first femoral implant has a first maximum overall exterior anterior to posterior dimension, a first lateral condyle, and a first medial condyle. The second femoral implant has a second maximum overall exterior anterior to posterior dimension that is different than the first maximum overall exterior anterior to posterior dimension, a second lateral condyle having a medial to lateral dimension that is substantially the same as a medial to lateral dimension of the first lateral condyle, and a second medial condyle having a medial to lateral dimension that is different than a medial to lateral dimension of the first medial condyle.

Another embodiment provides for a knee joint prosthesis system adapted to replace the articulating knee portion of a femur and a tibia, the femur having a resected engagement surface, the knee joint prosthesis system having a set of implants. The set of implants comprises a first femoral implant, a second femoral implant, a tibial implant, and a bearing member. The first femoral implant has a first maximum overall exterior anterior to posterior dimension, a first maximum interior anterior to posterior dimension, and a first condyle having a first posterior thickness. The second femoral implant has a second maximum overall exterior anterior to posterior dimension that is greater than the first maximum overall exterior anterior to posterior dimension, a second maximum interior anterior to posterior dimension that is substantially the same as the first maximum interior anterior to posterior dimension and a second condyle having a second posterior thickness that is greater than the first posterior thickness. The tibial implant has a tibial bearing surface. The bearing member is operable to be positioned between one of the first and the second femoral implants and the tibial implant.

Another embodiment provides for a kit for replacing portions of an articulating knee joint including a femur and a tibia, the femur having a resected engagement surface. The kit comprises a plurality of cruciate retaining femoral components, a first tibial component, a first bearing component, a plurality of posterior stabilized femoral components, a second tibial component, a second bearing component, a plurality of constrained femoral components, a third tibial component, and a third bearing component. The plurality of cruciate retaining femoral components each include a patella bearing portion, a medial condyle having a medial posterior region, and a lateral condyle having a lateral posterior region. The first bearing component is operable to engage the first tibial component and operable to articulate with at least one of the cruciate retaining femoral components. The plurality of posterior stabilized femoral components each include a patella bearing portion, a medial condyle having a medial posterior region, a lateral condyle having a lateral posterior region, and an intercondylar box. The second bearing component has a guide post. The second bearing component is operable to engage the second tibial component and articulate with at least one of the posterior stabilized femoral components. The plurality of constrained femoral components each include a patella bearing portion, a medial condyle having a medial posterior region, a lateral condyle having a lateral posterior region, and an intercondylar box. The third bearing component has a guide post and is operable to engage the third tibial component and articulate with at least one of the constrained femoral components. At least one of the femoral components includes a first overall outer anterior to posterior dimension, a first overall interior anterior to posterior dimension, and a first posterior region of the medial and lateral condyles that are unitary with the femoral component and at least one of the femoral components include a second overall outer anterior to posterior dimension, a second overall interior anterior to posterior dimension, and a second posterior region of the medial and lateral condyles that are unitary with the femoral component. The first overall outer anterior to posterior dimension and the second overall outer anterior to posterior dimension are different and the first overall interior anterior to posterior dimension and the second overall interior to posterior dimension are substantially the same. At least one of the femoral components include a third overall interior anterior to posterior dimension and a third overall medial to lateral dimension, and a third posterior region of the medial and lateral condyles that are unitary with the femoral component, and at least one of the femoral components include a fourth overall interior anterior to posterior dimension and a fourth overall medial to lateral dimension, and a fourth posterior region of the medial and lateral condyles that are unitary with the femoral component. The third overall interior anterior to posterior dimension and the fourth overall interior anterior to posterior dimension are substantially the same, and the third overall medial to lateral dimension and the fourth overall medial to lateral dimension are different.

### BRIEF DESCRIPTION OF THE DRAWINGS

Still other advantages of the present invention will become apparent to those skilled in the art after reading the following specification and by reference to the drawings in which:

FIG. 1 is a perspective view of a natural anatomical knee joint;

FIG. 2a is an exploded side view of a cruciate knee joint prosthesis according to the teachings of the present invention;

FIG. 2b is an exploded side view of a posterior stabilized (PS) knee joint prosthesis according to the teachings of the present invention;

FIG. 2c is an exploded side view of a fully constrained knee joint prosthesis according to the teachings of the present invention;

FIG. 3 is a perspective view of the cruciate knee joint according to the teachings of the present invention;

FIG. 4 is an assembled side view of the cruciate knee joint according to the teachings of the present invention;

FIG. 5 is a perspective view of the posterior stabilized (PS) knee joint prosthesis according to the teachings of the present invention;

FIG. 6 is an assembled side view of the posterior stabilized (PS) knee joint prosthesis according to the teachings of the present invention;

FIG. 7 is a front view of the posterior stabilized (PS) knee joint prosthesis according to the teachings of the present invention;

FIG. 8 is a perspective view of the fully constrained knee joint prosthesis according to the teachings of the present invention;

FIG. 9 is an assembled side view of the fully constrained knee joint prosthesis according to the teachings of the present invention.

FIG. 10a is a top view of the femoral component of FIG. 2a, the femoral component having a first medial/lateral width;

FIG. 10b is a posterior view of the femoral component of FIG. 10a;

FIG. 11a is a top view of the femoral component of FIG. 2a, the femoral component having a second medial/lateral width;

FIG. 11b is a posterior view of the femoral component of FIG. 11a;

FIG. 12a is a top view of a femoral component similar to that of FIG. 2a, the femoral component having a third medial/lateral width providing added width on a medial side of the component only; and

FIG. 12b is a posterior view of the femoral component of FIG. 12a.

### DETAILED DESCRIPTION OF THE PREFERRED EMBODIMENTS

The following description of the embodiment(s) directed to providing an elongated femoral component for the posterior stabilized and the fully constrained knee joint prosthesis are merely exemplary in nature and are not intended to limit its application or uses. The present invention is described in detail below with respect to floating bearing type knee joint prostheses. However, those skilled in the art will appreciate that the present invention is clearly not limited to only knee joint prostheses that employ floating bearings, but may be employed with any other type of knee joint prosthesis, such as a fixed bearing knee joint prosthesis.

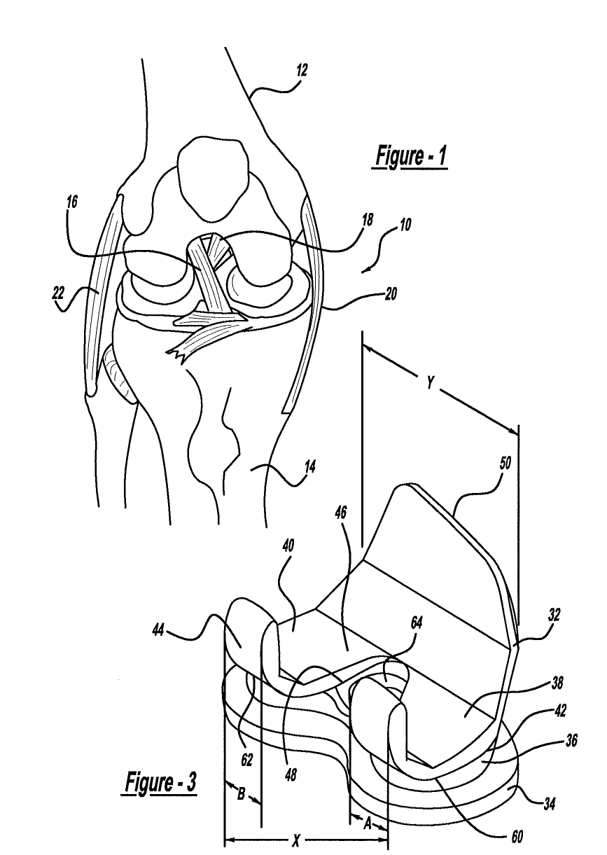

Referring to FIG. 1, an anatomical knee joint 10 which provides articulating motion between a femur 12 and a tibia 14 is illustrated in detail. The anatomical knee joint 10 includes a posterior cruciate ligament (PCL) 16 and an anterior cruciate ligament (ACL) 18, which cross each other within the knee joint 10. The PCL and ACL ligaments 16 and 18 provide stability and strength to the knee joint 10. The knee joint 10 also includes a medial collateral ligament 20 and a lateral collateral ligament 22 used to balance the medial and lateral forces in the knee joint 10.

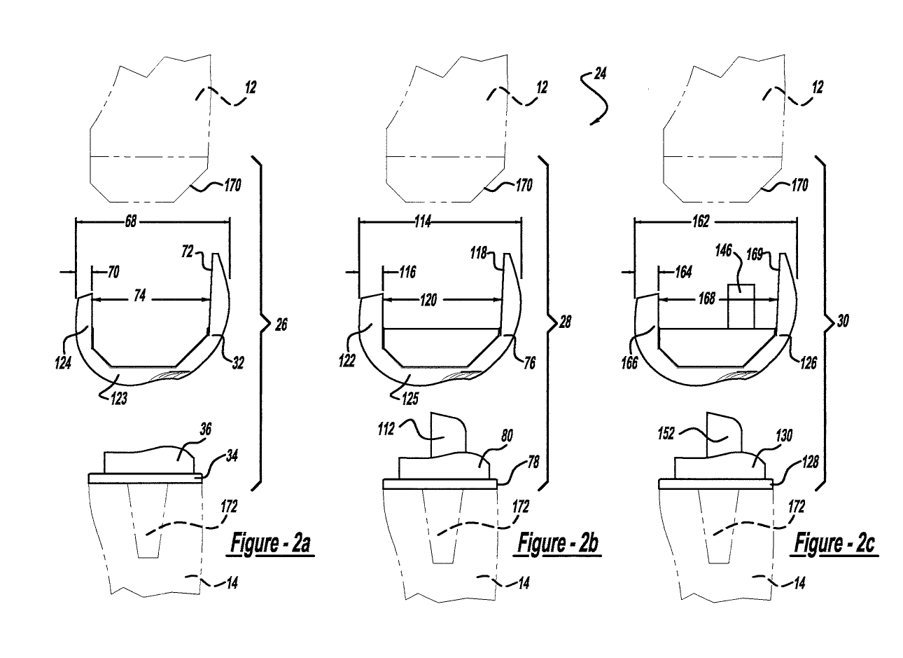

With reference to FIGS. 2a-2c, the knee joint prosthesis system 24 according to the teachings of the present invention is shown. The knee joint prosthesis system 24 generally includes a kit having a cruciate retaining (CR) knee joint prosthesis 26 (see FIG. 2a), a posterior stabilized (PS) knee joint prosthesis 28 (see FIG. 2b), and a fully constrained knee joint prosthesis 30 (see FIG. 2c). Each knee joint prostheses in the knee joint prosthesis system 24 provides a varying level of constraint with each being selected depending upon the patient's needs and the anatomical make-up of the particular patient. In this regard, should the anterior and posterior cruciate ligaments 18 and 16 not be sacrificed during the surgical procedure, a cruciate retaining (CR) knee joint prosthesis 26 will generally be employed which provides the least level of constraint. Should the anterior and posterior cruciate ligaments 18 and 16 be sacrificed or are dysfunctional and the medial and lateral collateral ligaments 20 and 22 remain functionally intact, the posterior stabilized (PS) knee joint prosthesis 28 will typically be employed which provides a higher level of constraint. Should both the anterior and posterior cruciate ligaments 18 and 16 be sacrificed and the medial or lateral collateral ligaments 20 and 22 be sacrificed or dysfunctional, the fully constrained knee joint prosthesis 30 will typically be employed, which provides the greatest level of constraint due to the sacrifice or loss of additional soft tissue ligaments.

It should be understood that the novel features of the knee joint prosthesis system 24 may be applied to any number of knee joint prostheses currently in existence, such as a fixed bearing or floating bearing type knee. For example, a fixed bearing type knee, such as that disclosed in U.S. Pat. No. 5,330,534, may employ the novel features of the present invention and is hereby incorporated by reference. Should a floating bearing knee joint prosthesis be employed, the floating bearing knee as disclosed in U.S. Pat. No. 6,165,223 or the floating bearing knee as disclosed in U.S. Pat. No. 6,413,279, issued Jul. 2, 2002, entitled “Floating Bearing Knee Joint Prosthesis With A Fixed Tibial Post” may also be employed, each of which are hereby incorporated by reference.

Accordingly, the novel features disclosed with the knee joint prosthesis 24 may be employed with any type of knee joint prosthesis currently in existence to provide a surgeon with interoperative options for selection of a proper level of constraint, while addressing the concern of an increased flexion gap should the posterior cruciate ligament 16 be sacrificed. This selectability will also be available without the need for further bone resection once the femur 12 has been properly resected for fitting with trial prostheses, which are known in the art and will be further discussed herein. In this regard, the present knee joint prosthesis system 24 provides equal distal and posterior condyle thickness' for the cruciate retaining knee joint prosthesis 26, but within the same system 24, the posterior stabilized knee joint prosthesis 28 and the fully constrained knee joint prosthesis 30 provide a thicker posterior condyle thickness than the distal condyle thickness. This provides the advantage of equalizing the collateral ligament tensions from extension to flexion after the PCL 16 is removed. This compares to current practices where a surgeon will typically “upsize” the femoral component and maintain the anterior reference and the anterior placement of the component, thereby oftentimes providing a femoral component that is too large in the medial to lateral dimension which may over extend the femur 12.

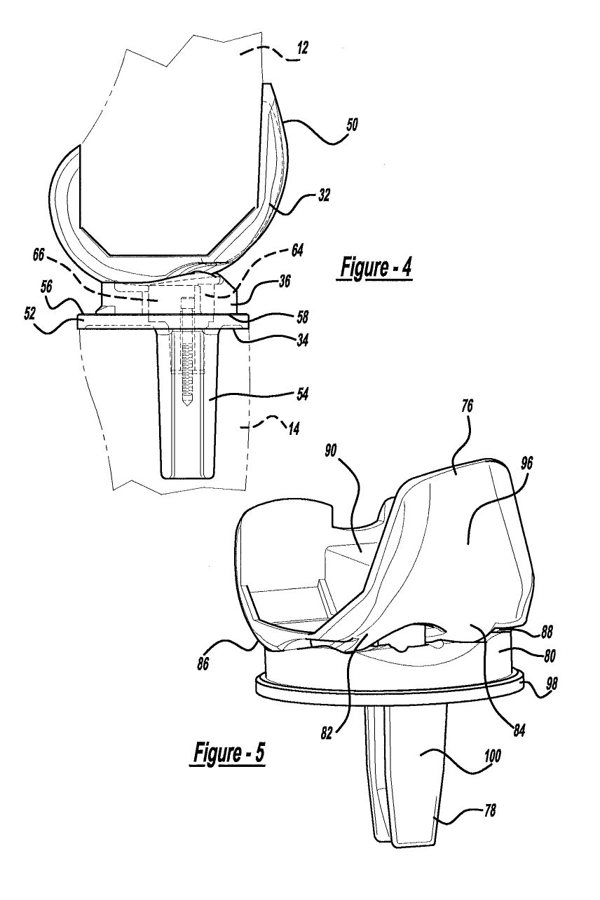

Referring now to FIGS. 2a, 3 and 4, the cruciate retaining knee joint prosthesis 26 will be discussed in detail. The cruciate retaining (CR) knee joint prosthesis 26 is shown to be secured to the femur 12 and the tibia 14 of a surgically resected left knee joint, with the femur 12 and the tibia 14 shown in phantom, and with the understanding that a suitable right knee joint prosthesis can be similarly constructed. The cruciate retaining (CR) knee joint prosthesis 26 includes a femoral component 32, a tibial component 34 and a floating tibial bearing 36.

The femoral component 32 is adapted to be secured to the distal end of the femur 12 and includes a first condylar portion 38 and a second condylar portion 40. The first condylar portion 38 is illustrated as a medial condyle and the second condylar portion 40 is illustrated as a lateral condyle. The first condyle portion 38 and the second condylar portion 40 each provide a first femoral bearing surface 42 and a second femoral bearing surface 44 respectively. The first and second condylar portions 38 and 40 of the femoral component 32 are interconnected by an intercondylar portion 46 that defines an inner condylar opening 48 to accommodate the posterior and anterior cruciate ligaments 16 and 18. The femoral component 32 also includes an arcuate patellar portion 50, which is disposed on the anterior surface of the femoral component 32. The patellar portion 50 is shaped to allow anatomical tracking of a natural or a prosthetic patella. The patella prosthesis, which are compatible with the present invention may be of varying shape, such as round or dome shaped and may be constructed from polyethylene, polyethylene with metal backing, or other suitable materials. The femoral component 32 is formed as a unitary structure and preferably cast of a biocompatible high strength alloy, such as cobalt-chromium-molybdenum alloy or other suitable material. All surfaces, which do not contact the femur 12, are preferably highly polished to provide smooth articulating bearing surfaces.

The tibial component 34 is adapted to be secured to the proximal end of the tibia 14 after the tibia 14 has been resected in a manner known in the art. The tibial component 34 includes a substantially planar platform-like tibial tray 52 and an inferior extending tibial stem 54. The tibial stem 54 is adapted to be received in a corresponding opening made by a surgeon in the longitudinal center of the tibial 14. The tibial tray 52 and the tibial stem 54 can be manufactured from cobalt-chromium-molybdenum or other suitable biocompatible material. The top of the tibial tray 52 is highly polished to provide a substantially smooth tibial bearing surface 56.

The floating or rotating bearing 36 is located between the femoral component 32 and the tibial component 34. The floating bearing 36 has a substantially planar inferior bearing surface 58, which slidably moves relative to the highly polished tibial bearing surface 56. The floating bearing 36 also includes a first superior articulating or bearing surface 60 and a second superior articulating or bearing surface 62. The first bearing surface 60 and the second bearing surface 62 articulate with the first bearing surface 42 of the first condylar portion 38 and the second bearing surface 44 of the second condylar portion 40 of the femoral component 32. Positioned between the first and second bearing surfaces 60 and 62 is a substantially rectangular opening 64 that is slidably positioned about a center modular guide post 66 which is secured to the tibial tray 52 by any appropriate means, such as a Morse taper with a screw. The opening 64 and the post 66 are sized to permit anterior to posterior, medial to lateral and rotational movement of the bearing 36. The center guide post 66 may not extend beyond the bearing 36 and can be formed from a combination of a cobalt-chromium-molybdenum portion and a molded polymer portion formed from UHMWPE or other suitable material. The bearing 36 can be formed from a surgical grade, low pressure, low wearing plastic, such as UHMWPE or other suitable material.

The femoral component 32 has an overall anterior to posterior dimension 68 and a posterior condyle thickness 70, further discussed herein. The femoral component 32 is also referenced from the anterior point 72 to provide an anterior to posterior mating area dimension or interior anterior to posterior dimension 74. This dimension 74 extends from the anterior point 72 to a posterior condylar region 124 and corresponds to elongated lateral cuts made to the femur 12 rather than an overall interior mating shape that corresponds to cuts made at a resected engagement surface 170 of the femur 12. This overall mating shape may be of various different shapes to correspond to the various different shapes of the engagement surface 170 or of the same shape as illustrated.

As was previously indicated, the cruciate retaining (CR) femoral knee joint prosthesis 26 is generally employed when the anterior and posterior cruciate ligaments 18 and 16 are retained during a surgical procedure and therefore provides the least level of constraint. By retaining the posterior cruciate ligament 16, the flexion gap generally does not increase between the femur 12 and the tibia 14 during flexion and the extension gap also does not undergo any noticeable change.

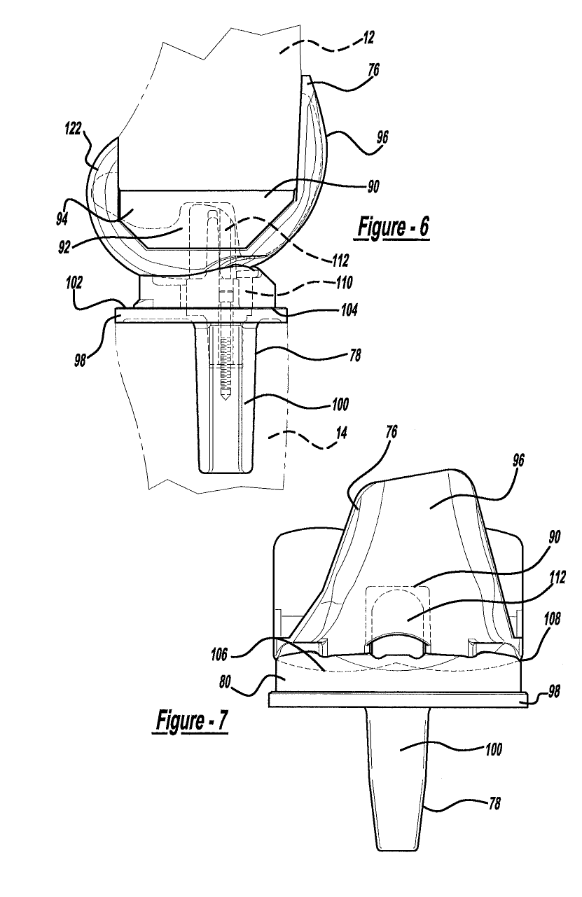

Referring now to FIGS. 2b and 5-7, the posterior stabilized (PS) knee joint prosthesis 28 is shown and discussed in further detail. Here again, the posterior stabilized (PS) knee joint prosthesis 28 is generally employed when the anterior and posterior cruciate ligaments 18 and 16 are sacrificed, thereby providing an additional level of constraint due to loss of these particular ligaments.

The posterior stabilized (PS) knee joint prosthesis 28 includes a femoral component 76, a tibial component 78 and a bearing component 80. Here again, the femoral component 76 includes a first condylar portion 82, a second condylar portion 84 having a first bearing surface 86 and a second bearing surface 88, respectively. The first condylar portion 82, is illustrated, as a medial condyle and the second condylar portion 84, is illustrated, as a lateral condyle. The first and second condylar portions 82 and 84 are interconnected by an intercondylar portion 90 that defines an intercondylar recess 92 and a cam or lobe 94. The intercondylar portion 90 essentially defines an intercondylar box that houses the lobe or cam 94, further discussed herein. The femoral component 76 also includes an arcuate patellar portion 96 that is disposed on the anterior surface of the femoral component 76, which is shaped to allow anatomical tracking of a natural or a prosthetic patellar. Here again, the femoral component 76 can be formed as a unitary structure and preferably cast of a biocompatible high strength alloy, such as cobalt-chromium-molybdenum alloy or other suitable material. All surfaces that do not contact the femur 12, can be highly polished to provide smooth articulating bearing surfaces.

The tibial component 78 is substantially similar or the same as the tibial component 34 and is adapted to be secured to the proximal end of the tibia 14 after the tibia 14 has been resected in a manner known in the art. The tibial component 78 includes a substantially planar platform-like tibial tray 98 and an inferiorly extending tibial stem 100. The tibial stem 100 is again adapted to be received in a corresponding opening made by a surgeon in a longitudinal center of the tibia 14 and the tibial tray 98 is highly polished to provide a substantially smooth tibial bearing surface 102. The tibial component 78 can be manufactured from cobalt-chromium-molybdenum or other suitable biocompatible material.

The floating or rotating bearing 80 is positioned between the femoral component 76 and the tibial component 78. The floating bearing 80 includes a substantially planar inferior bearing surface 104 and first and second superior articulating bearing surfaces 106 and 108. Located between the first and second bearing surfaces 106 and 108 is a substantially rectangular opening 110 that is slidably positioned about a center modular guide post 112 which extends up through the rectangular opening 110. The opening 110 provides for anterior to posterior, medial to lateral, and rotational movement of the bearing 80 relative to the tibial tray 98, but is somewhat more constrained than the opening 64 associated with the cruciate retaining knee joint prosthesis 26. Here again, the bearing 20 can be formed from UHMWPE or other suitable material.

The center guide post 112 has a substantially oval shaped outer peripheral sidewall or any other appropriately shaped sidewall that enables anterior to posterior, as well as medial to lateral and rotational movement of the bearing 80 relative to the tibial tray 98. Also, the center guide post 112 extends superiorly into the intercondylar recess 92 of the intercondylar portion 90 to engage the cam or lobe 94 during flexion. In this regard, when the posterior stabilized (PS) knee joint prosthesis 28 is in flexion, the engagement of the guide post 112 relative to the lobe or cam 94 forces the bearing component 80, as well as the femoral component 76 to provide for a more natural anatomical femoral rollback, as discussed in detail in U.S. Pat. No. 6,165,223, and as disclosed in U.S. Pat. No. 6,413,279, issued Jul. 2, 2002, entitled “Floating Bearing Knee Joint Prosthesis With A Fixed Tibial Post”, which are each hereby incorporated by reference.

Referring back to FIG. 2b, the femoral component 76 includes an overall anterior to posterior dimension 114 and a posterior condyle thickness 116. The femoral component 76 further includes an anterior reference point 118 and an anterior to posterior mating area dimension or interior anterior to posterior dimension 120. This dimension 120 extends from the anterior point 118 to a posterior condylar region 122 and corresponds to elongated lateral cuts made to the femur 12 rather than an overall internal mating shape that corresponds to cuts made at a resected engagement surface 170 of the femur 12. This overall mating shape may be of various different shapes to correspond to the various different shapes of the engagement surface 170 or of the same shape as illustrated.

A posterior condylar region 122 of the femoral component 76 has a substantially larger posterior condylar thickness 116 as compared with the posterior condylar region 124 of the femoral component 32 which has the posterior condylar thickness 70. The posterior thickness 70 is about 8 mm, while the posterior thickness 116 is about 8 to 16 mm. The distal condylar thickness of the distal regions 123 and 125 of the cruciate retaining (CR) femoral component 32 and the posterior stabilized (PS) femoral component 76 are substantially the same. Also, the posterior condylar thickness 70 of the cruciate retaining (CR) femoral component 32 is substantially the same as the distal condylar thickness of the distal region 123.

During a surgical procedure, should a surgeon pre-operatively determine to use the cruciate retaining (CR) knee joint prosthesis 26, the surgeon will typically resect the tibia 14 and implant the tibial component 34. The surgeon will then generally resect the femur 12 and either uses a trial component (not shown) or the femoral component 32 having the anterior to posterior resection or mating area dimension 74. If, during the surgical procedure, a surgeon determines that the posterior cruciate ligament 16 must be sacrificed, the femoral component 32 will not provide a large enough thickness in the posterior condylar region 124 to accommodate for the increased flexion gap by removal of the posterior cruciate ligament 16. To accommodate for this interoperative decision, the posterior stabilized (PS) femoral component 76 may simply be utilized in place of the cruciate retaining (CR) femoral component 32 since the anterior to posterior resection or mating area dimension 120 of the femoral component 76 is substantially identical to the anterior to posterior resection or mating area dimension 74 of the femoral component 32.

In this regard, the anterior reference points 72 and 118 of each femoral component 32 and 76 is the same with the posterior stabilized (PS) femoral component 76 providing a thicker posterior condyle portion 122, identified as reference numeral 116 that extends further posteriorly. Therefore, the anterior to posterior resection or mating area dimension 74 and 120 are substantially the same, with the understanding that a femoral cut to accommodate the intercondylar portion 90 may be required. Likewise, the guide post 66 in the tibial component 34 may be simply replaced with the guide post 112, as well as the bearing 80 in place of the bearing 32, which provides for a smaller opening 110 providing a further level of constraint. This way, a surgeon can simply interoperatively select either the cruciate retaining (CR) knee joint prosthesis 26 or the posterior stabilized (PS) knee joint prosthesis 28 without having to perform any further adjustments or anterior to posterior resections to the femur 12 or tibia 14. The only femoral cut that may be necessary is to accommodate for the intercondylar portion 90. In this way, the flexion gap typically associated with PCL removal is adequately accommodated with the posterior stabilized (PS) femoral component 76. In contrast, with existing systems a surgeon would typically either require a posterior condylar augment by upsizing the femoral component, such that there may be medial and lateral overhang of the component relative to the femur 12, which is an undesirable condition, or further anterior to posterior cuts may be required.

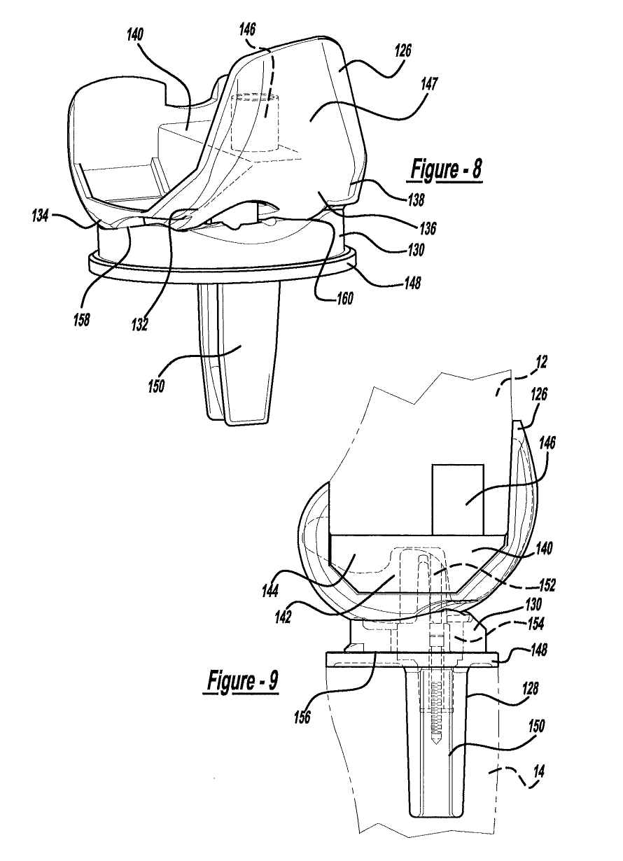

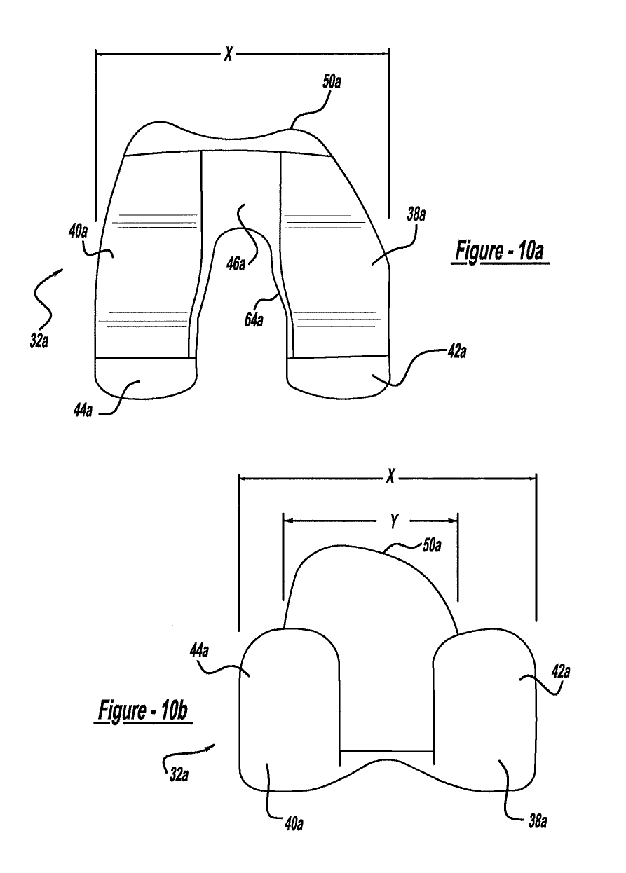

Finally, referring to FIGS. 2c, 8 and 9, the fully constrained knee joint prosthesis 30 is shown in further detail. In this regard, the fully constrained knee joint prosthesis system 30 is substantially similar to the posterior stabilized (PS) knee joint prosthesis 28 except for a few slight modifications. The fully constrained knee joint prosthesis 30 again includes a femoral component 126, a tibial component 128 and a bearing 130. The femoral component 126 again includes a first condylar portion 132 having a first bearing surface 134 and a second condylar portion 136 having a second bearing surface 138. The first condylar portion 132, is illustrated, as a medial condyle and the second condylar portion 136, is illustrated, as a lateral condyle. An intercondylar portion 140 interconnects the first and second condylar portions 132 and 134 which also defines an intercondylar recess 142 having a cam lobe 144. Extending superiorly from the intercondylar portion 140 is a substantially cylindrical stem 146, which provides further support for the fully constrained femoral component 126. A patella surface 147 is also provided anteriorly of the femoral component 126.

The tibial component 128 is the same or substantially similar to the tibial components 34 and 78 and again includes a tibial plateau 148, and an inferior extending stem 150. Removably attached to the tibial component 128 is a guidepost 152, which extends up through a recess 154 in the bearing 130. The bearing 130 again includes the inferior bearing surface 156, and the first and second superior articulating bearing surfaces 158 and 160.

The fully constrained knee joint prosthesis 30 is substantially similar to the posterior stabilized knee joint prosthesis 28, except that it provides the further stabilizing cylindrical stem 146, as well as provides and defines a smaller opening 154 within the bearing 130. This smaller opening 154 engages the medial and lateral sidewalls of the guide posts 152 to provide for only anterior to posterior movement of the bearing 130 relative to the tibial plateau 148. The medial to lateral constraint is provided since the medial and collateral ligaments 20 and 22 are generally removed when the fully constrained knee joint prosthesis 30 is employed.

Here again, the femoral component 126 includes an anterior to posterior dimension 162 and posterior condyle thickness 164 for the posterior condylar portion 166 and an anterior to posterior mating area dimension or interior anterior to posterior dimension 168. This dimension 168 extends from anterior reference point 169 to posterior condylar region 166 and corresponds to elongated lateral cuts made to the femur 12 rather than an overall internal mating shape which corresponds to cuts made at a resected engagement surface 170 of the femur 12. This overall mating shape may be of various different shapes to correspond to the various different shapes of the engagement surface 170 or of the same shape as illustrated.

The anterior to posterior dimension 162, the posterior thickness 164, the anterior to posterior resection or mating area dimension 168, and the distal condylar thickness are substantially the same as the corresponding dimensions in the posterior stabilized femoral component 76.

Additionally, it should be noted that the anterior to posterior resection or mating area dimension 74, 120 and 168 are substantially the same for each knee joint prosthesis 26, 28 and 30, along with the distal condylar thickness. In this regard, the posterior stabilized (PS) knee joint prosthesis 28 and the fully constrained knee joint prosthesis 30 can provide additional posterior thickness 116 and 164 which are greater than the posterior thickness 70 in the cruciate retaining (CR) knee joint prosthesis 26 to accommodate for the increased flexion gap. This increased thickness comes as a result of adding additional material posteriorly, as opposed to providing further removal of femoral bone, thereby enabling the anterior to posterior resecting mating area or dimension for each knee joint prosthesis to be substantially the same, as well as the distal condylar thickness of each knee joint prosthesis. This enables a surgeon to interoperatively select between either the cruciate retaining (CR) knee joint prosthesis 26, the posterior stabilized (PS) knee joint prosthesis 28 or the fully constrained knee joint prosthesis 30 without having to further resect the femur 12, except to account for the intercondylar portions 90 and 140, and also provide for a thicker posterior condyle region to accommodate for flexion gap increases should the posterior cruciate ligament 16 be removed. This provides the advantage of equalizing the collateral ligament tensions in the posterior stabilized (PS) knee joint prosthesis 30 from extension to flexion after the posterior cruciate ligament 16 is surgically removed. This also maintains the anterior reference point and the anterior placement of each femoral component.

To accommodate femurs 12 having different medial/lateral (ML) widths, such as between male and female patients, without changing either the overall anterior to posterior (A/P) dimension or the overall interior A/P dimension at the engagement region, the cruciate retaining (CR) femoral component 32, the PS femoral component 76, and the fully constrained femoral component 126 may each be provided with varying medial/lateral (ML) dimensions. For example, a crucriate retaining (CR) femoral component having an enlarged M/L dimension is illustrated in FIGS. 10a and 10b at 32a. The CR femoral component 32a is substantially similar to the CR femoral component 32 and elements in common between the CR femoral component 32 and the CR femoral component 32a are designated with like reference numbers bearing the letter “a.” As the CR femoral component 32a is substantially similar to the CR femoral component 32, the detailed description of the CR femoral component 32 also applies to the CR femoral component 32a and need not be repeated here.

The only substantial difference between the CR femoral component 32a and the CR femoral component 32 is that the CR femoral component 32a has an overall M/L dimension X, and an arcuate patellar portion 50a M/L dimension Y, that is greater than an overall M/L dimension X of the CR femoral component 32 and the M/L dimension Y of the arcuate patellar portion 50 without substantially changing the overall interior A/P dimension 74 or the resected engagement region. Specifically, the overall M/L dimension of the of the CR femoral component 32a from the first condylar portion 38a to the second condylar portion 40a and the M/L dimension of the arcuate patellar portion 50a is greater than that of the CR femoral component 32. While only the CR femoral component 32 is illustrated as having an increased M/L dimension, the PS femoral component 76 and the fully constrained femoral component 126 may also be provided with increased M/L dimensions to accommodate larger femurs 12 without substantially changing the overall A/P dimension or the A/P mating area dimension.

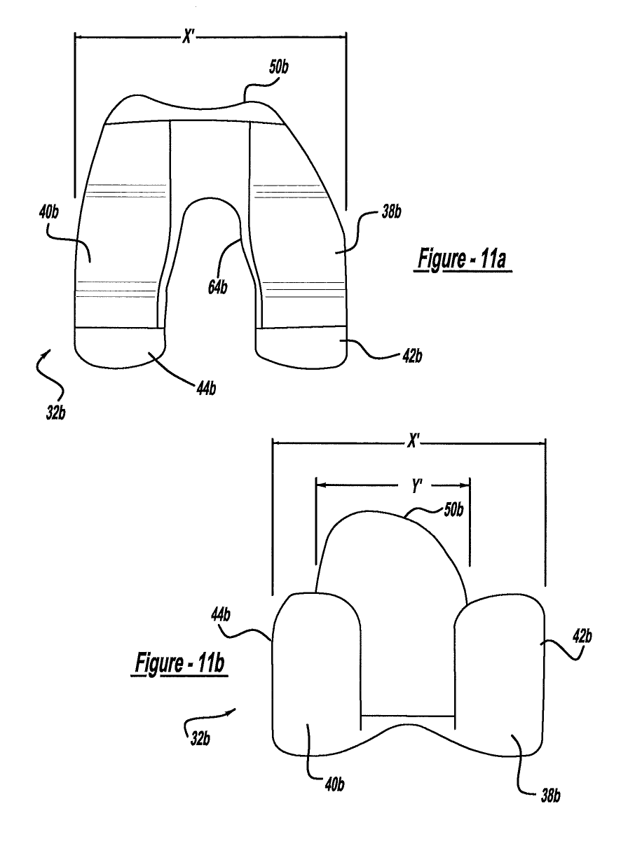

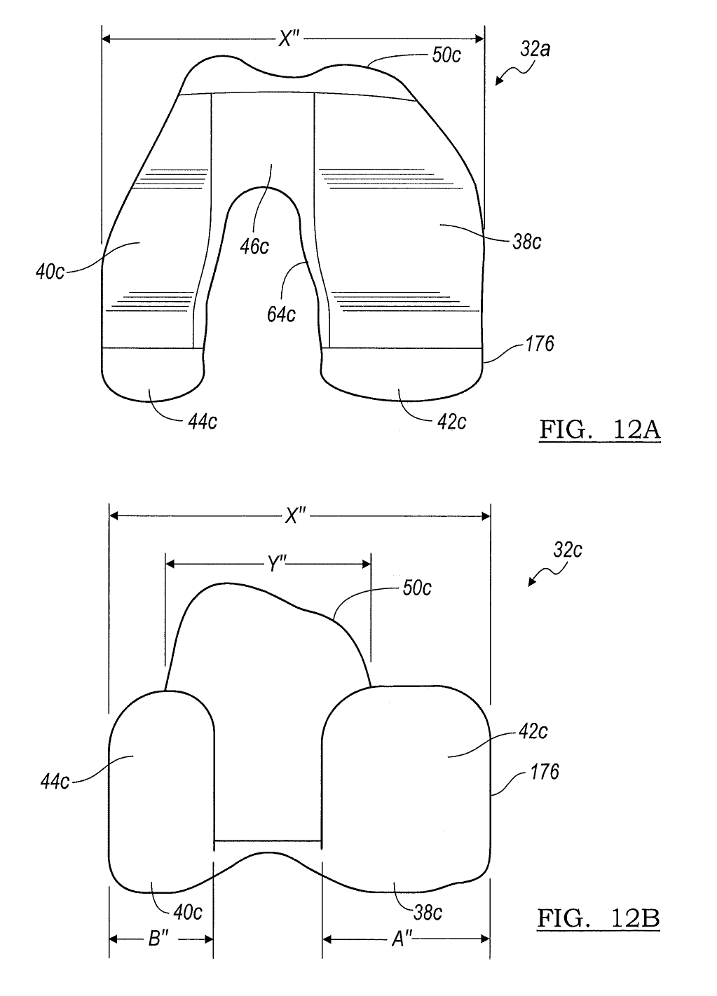

To accommodate femurs 12 having smaller M/L dimensions, the cruciate retaining (CR) femoral component 32, the PS femoral 76, and the fully constrained femoral component 126 may each be provided with smaller M/L dimensions without substantially changing the overall A/P dimensions or the overall interior A/P dimensions of the resected engagement regions. For example, a CR femoral component 32b having an M/L dimension X′ that is smaller than the M/L dimension of the CR femoral component 32 may be used, and is illustrated in FIGS. 11a and 11b. The CR femoral component 32b is substantially similar to the CR femoral component 32 and is described with like reference numbers that bear the letter “b.” The only substantial difference between the CR femoral component 32 and the CR femoral component 32b is that the overall M/L dimension X′, and M/L dimension Y′ of the arcuate patellar portion 50b, of the CR femoral 32b is smaller than the overall ML dimension X, and the M/L dimension Y of the arcuate patellar portion 50, of the CR femoral component 32. While only the CR femoral component 32 is illustrated as having a smaller M/L dimension X′, the PS femoral component 76 and the fully constrained femoral component 126 may also be provided with decreased M/L dimensions to accommodate smaller femurs 12 without substantially changing the total A/P dimension or the dimension of the resected engagement regions.

To accommodate femurs 12 having a greater maximum overall medial condyle dimension, the cruciate retaining femoral component 32, the PS femoral component 76, and the fully constrained femoral component 126 may each be provided with a medial condyle and an arcuate patellar portion that are each wider at the medial side, without substantially changing the overall exterior A/P dimensions or the overall interior A/P dimensions. For example and with reference to FIGS. 12A and 12B, a CR femoral 32c having an overall M/L dimension X″ that is larger than the overall M/L dimension X of the CR femoral 32 is illustrated. While a CR femoral component is illustrated, a PS femoral component and a fully constrained femoral component may also have this configuration.

The CR femoral component 32c includes a medial condyle 38c having an overall maximum medial/lateral dimension A″ that is greater than an overall maximum medial/lateral dimension A of the medial condyle 38 of the CR femoral component 32. Further, the CR femoral component 32c includes an arcuate patellar portion 50c that has a medial/lateral dimension Y″ that is greater than a medial/lateral dimension Y of the arcuate patellar portion 50 of the CR femoral component 32. It is the enlarged medial/lateral dimensions of the medial condyle 38c and the arcuate patellar portion 50c that provide the CR femoral component 32c with an M/L dimension that is greater than the M/L dimension of the CR femoral component 32. The medial/lateral dimension B″ of the lateral condyle 40c of the CR femoral component 32c is substantially the same as the medial/lateral dimension B of the lateral condyle 40 of the CR femoral component 32. The CR femoral component 32c is substantially similar to the CR femoral component 32 and is described with like reference numbers that include the letter “c.”

The only substantial difference between the CR femoral component 32 and the CR femoral component 32c is that the overall M/L dimension X″ and the arcuate patellar M/L dimension Y″ of the CR femoral component 32c is larger at medial side 176 than the overall M/L dimension X and the M/L arcuate patellar dimension Y of the CR femoral 32. While only the CR femoral component 32c is illustrated as having larger M/L dimensions X″ and Y″, the PS femoral component 76 and the fully constrained femoral 126 may also be provided with similar increased M/L dimensions at the medial side to accommodate larger medial condyles without substantially changing the overall exterior A/P dimensions or the overall interior A/P dimensions. The present teachings give a surgeon the ability to choose a femoral component for a knee joint prosthesis from a variety of different femoral components, such as the CR femoral components 32a, 32b, and 32c that have a varying overall M/L dimension and a constant overall interior A/P dimension allowing constant femoral engagement. Conventionally, if a surgeon sizes a patient for a femoral component of a first size, rejoining the femur based on that size, but later determines that a wider or narrower femoral component is required due to the width of the femur, the surgeon will simply select a femoral component having a larger or smaller overall size. However, the use of a femoral component of a different overall size will require the surgeon to re-cut the femur. Additionally, by selecting the proper overall M/L or wider M/L dimension, the A/P dimension is then typically either too large or too small. The present invention eliminates the need to re-cut the femur, when it is determined that a femoral component of a different overall M/L width is required, by providing the surgeon with multiple femoral components that are similar in most all respects, except for their M/L widths.

In operation, preoperatively, the surgeon will select whether to use the cruciate retaining (CR) knee joint prosthesis 26, the posterior stabilized (PS) knee joint prosthesis 28, or the fully constrained knee joint prosthesis 30. If the surgeon has pre-selected the cruciate retaining (CR) knee joint prosthesis 26, the end of the femur 12 will be resected and the surgeon will form a resected engagement surface 170 with the intention of implanting the first femoral component 32 having a corresponding anterior to posterior resection or mating area dimension 74. The end of the tibia 14 is also resected to cooperatively engage the tibial component 34. A passageway 172 is also formed in the tibia 14 and the stem 54 is inserted into the passageway 172 as is conventional. The bearing 36 is placed between the tibial tray 52 and the first femoral component 32.

If interoperatively in trial, the surgeon determines that the natural knee joint 10 is found to be more severely deformed or dysfunctional than it was preoperatively thought to be, the surgeon must make a decision about whether or not to resect the posterior cruciate ligament (PCL) 16. Some surgeons will attempt to preserve the posterior cruciate ligament (PCL) 16 if at all possible, while others prefer preservation in only selected cases where there is less deformity in the knee joint 10. If satisfactory soft tissue balance cannot be obtained, the surgeon will consider changing from a cruciate retaining (CR) knee joint prosthesis 26 to a posterior stabilized (PS) knee joint prosthesis 28. If the surgeon decides not to resect the PCL 16, the surgeon will continue with the procedure and retain the first femoral component 32, the bearing 36 and the tibial component 34, in the knee joint 10 as is conventional.

If the posterior cruciate ligament 16 is resected, the surgeon will be confronted with the need to compensate for an increase in flexion gap. To compensate for the increase in flexion gap, the surgeon will remove the first (CR) femoral component 32 and instead implant the second (PS) femoral component 76. Since the anterior to posterior resection or mating area dimension 120 of the second femoral component 70 is the same as the anterior to posterior resection or mating area dimension 74 of the first femoral component 32, the surgeon will not be required to resect the end of the femur 12 as the resected engagement surface 170 will cooperatively engage the anterior to posterior resection or mating area dimension 120. Only resection to accommodate for the intercondylar portion 90 will be required, which does not change the shape of the resected engagement surface 170. This saves time and reduces the complexity of the surgical procedure for the surgeon.

The second femoral component 76 has the larger posterior condylar thickness 116 than a distal condylar thickness 174 in order to compensate for the increase in flexion gap which could be between 4 mm to 8 mm. The required size of the posterior condylar thickness 116 is selected by the surgeon to balance the flexion and extension in the knee joint 10. The posterior condylar thickness 116 is in the range of about 8 to 16 mm and the distal condylar thickness 174 is between about 8 to 9 mm. This eliminates the need to compromise the range of motion of the knee joint prosthesis or require further surgical resectioning of the femoral engagement surface.

Regardless of whether the physician chooses the CR femoral component 32, the PS femoral component 76, or the fully constrained femoral component 126, the femoral component chosen must have an M/L width that matches the M/L width of the patient's femur. For example, if the physician determines that the CR femoral 32 is required, the physician must then determine whether the M/L width of the patient's femur requires the standard CR femoral component 32, the CR femoral component 32a having the enlarged M/L width, the CR femoral component 32b having the narrow M/L width, or the CR femoral component 32c having an enlarged M/L width on the medial side only. Regardless of whether the CR femoral component 32, 32a, 32b, or 32c is initially chosen, the physician need not re-cut the femur 12 if the physician later decides to use a different CR femoral component 32, 32a, 32b, or 32c of a different M/L width because the overall exterior A/P dimension 68 of each of the CR femoral components 32, 32a, 32b, and 32c is constant, as is the overall interior A/P dimension 74, thus providing a constant resected engagement region across the different CR femoral components 32, 32a, 32b and 32c. Further, because the A/P dimensions and the resected engagement regions of the CR femoral component 32, the PS femoral component 76, and the fully constrained femoral component 126 are substantially constant, regardless of the M/L widths of the femoral components 32, 76, and 126, the physician may easily substitute different femoral components 32, 76, and 126 of various different widths without having to alter the shape of the femur 12, thus saving operating room time and reducing the complexity of the procedure.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

1.-7. (canceled)

8. A method for implanting a femoral implant of a knee joint prosthesis system having a set of femoral implants to a femur having a resected engagement surface, said method comprising:
resecting the femur to provide the resected engagement surface;
providing a first femoral implant having a first maximum interior anterior to posterior dimension, a first maximum overall exterior anterior to posterior dimension, a first lateral condyle, and a first medial condyle;
providing a second femoral implant having a second maximum interior anterior to posterior dimension that is substantially the same as said first maximum interior anterior to posterior dimension, a second maximum overall exterior anterior to posterior dimension that is different than said first maximum overall exterior anterior to posterior dimension, a second lateral condyle having a medial to lateral dimension that is substantially the same as a medial to lateral dimension of said first lateral condyle, and a second medial condyle having a medial to lateral dimension that is different than a medial to lateral dimension of said first medial condyle; and
securing either said first femoral implant or said second femoral implant to said femur.

9. The method of claim 8, wherein said first femoral implant includes a first arcuate patellar portion and said second femoral implant includes a second arcuate patellar portion having a medial to lateral dimension that is different than a medial to lateral dimension of said first arcuate patellar portion.

10. The method of claim 8, wherein one of said first femoral implant and said second femoral implant is secured to said femur without changing the resected engagement surface.

11. The method of claim 8, wherein the first femoral implant and the second femoral implant are selected from a group comprising a plurality of cruciate retaining femoral implants, a plurality of posterior stabilized femoral implants, and a plurality of constrained femoral implants.

12.-31. (canceled)
